(19) United States
(12) Patent Application Publication
(10) Pub. No.: US 2010/0036103 A1
(43) Pub. Date: Feb. 11, 2010

(54) Method for producing apoprotein

(75) Inventors: Hiroyoshi Inoue, Fukuoka (JP); Toshio Aritomi, Yamaguchi (JP); Minoru Kawashima, Yamaguchi (JP)

Correspondence Address: THE WEBB LAW FIRM, P.C., 700 KOPPERS BUILDING, 436 SEVENTH AVENUE, PITTSBURGH, PA, 15219, US

(73) Assignee: Kurume University, Fukuoka (JP)

(21) Appl. No.: 11/794,642

(22) PCT Filed: Dec. 28, 2005
(86) PCT No.: PCT/JP05/24286

§ 371 Date: Jul. 2, 2007

(30) Foreign Application Priority Data
Jan. 5, 2005 (JP) 2005-000343

(51) Int. Cl. C07K 1/34; B01J 19/00

(52) U.S. Cl. 530/395; 422/129

(57) ABSTRACT

A method and an apparatus for producing an apoprotein with which the apoprotein can be efficiently produced from a protein associated with cofactors such as metal ions. The method includes the step of adding an acid to a solution which contains a holoprotein, which is associated with a cofactor which is to be released under acidic conditions, and concentrating the solution with an ultrafiltration membrane. Accordingly, an apoprotein, which has released its cofactor, is produced, and the released cofactor is removed through the membrane along with the acid without reassociating with the apoprotein.

### TECHNICAL FIELD

The present invention relates to a method for producing an apoprotein from a holoprotein which is associated with a cofactor which is to be released under acidic conditions.

### BACKGROUND ART

Lactoferrin is a glycoprotein with a molecular weight of approximately 80,000 that has an ability to bind to iron, and has associated with two irons per molecule. Lactoferrin is found in bodily fluids, such as milk, of many mammals. In particular, it has been known that the colostrum contains 5 to 10 g/L of lactoferrin, accounting for 30 to 70% of the total proteins contained in the colostrum. Lactoferrin is not only an important protein for infant health and growth, but in recent years it has also been found to have antifungal and antibacterial activities as well.

Lactoferrin is generally extracted from colostrum, normal milk, cheese whey (the residue generated during the manufacture of cheese), and the like (for example, see Mamoru Tomita, MRC 19, 1997 pp. 106-108 and Mamoru Tomita, (1999) Foods Food Ingredients J. Jpn, 181:88-41).

For example, Mamoru Tomita, MRC 19, 1997 pp. 106-108 describes a method for obtaining a lactoferrin concentrate using the cationic property of lactoferrin. According to this method, whey is contacted with a cation exchange resin to adsorb lactoferrin to the cation exchange resin. Then, this resin is washed with a high-concentration salt solution to desorb lactoferrin, and the resultant solution which contains desorbed lactoferrin is desalted by ultrafiltration, yielding a lactoferrin concentrate. Other known methods for manufacturing a lactoferrin concentrate include the simple diffusion using a cation exchange membrane made of cellulose (Clovis K. Chiu and Mark R. Etzel, Journal of Food Science, 62(5), 1997, pp. 996-1001), the separation by electrophoresis (Hurly W L et al., J. Dairy Sci., 76, 1993, p. 377), the separation by affinity chromatography (M. K. Walsh and S. H. Nam, Prep. Biochem. Biotechnol., 31(3), 2001, pp. 229-240), and the separation by capillary electrophoresis (Peter Riechel et al., Journal of Chromatography A, 817, 1998, pp. 187-193).

In general, the extracted lactoferrin has 20 to 40% of irons associated. It has been known that apolactoferrin, which is obtained by removing irons from lactoferrin, has improved bacteriostatic activities compared with lactoferrin. When apolactoferrin is added in the culture medium for microorganisms, apolactoferrin can take an iron element, which is required to the growth of microorganisms, from the medium by its chelating action, to inhibit the growth of microorganisms. Thus, it is likely that apolactoferrin exhibits effective bacteriostatic activities against any microorganisms that highly require an iron element for their growth.

Generally, apolactoferrin has been produced by batch methods. For example, apolactoferrin is produced by adding an acid such as hydrochloric acid or citric acid to the lactoferrin-containing liquid, which is obtained from whey or the like, to adjust the pH of liquid to approximately 2 and release iron from lactoferrin. However, as far as the released iron and apolactoferrin exist together in a liquid, they will reassociate during the extraction of lactoferrin, making it difficult to obtain apolactoferrin efficiently. On the purification of apolactoferrin, the anion of the added acid will be contaminant. Other methods for producing apolactoferrin include the method of dialyzing lactoferrin against a citric acid solution and the method of contacting lactoferrin with a chelating agent such as ethylenediaminetetraacetatic acid (EDTA). However, none of these are particularly efficient methods for producing apolactoferrin.

### DISCLOSURE OF INVENTION

Therefore, it is an object of the present invention to provide a method for producing an apoprotein with which the apoprotein can be efficiently produced from a protein associated with cofactors such as metal ions.

The invention provides a method for producing an apoprotein, which includes the step of adding an acid to a solution which contains a protein associated with a cofactor which is to be released under acidic conditions and applying them to ultrafiltration. By this step, an apoprotein which has released the cofactor is produced, and the released cofactor is removed through the membrane along with the acid.

In one embodiment, the step of adding acid and ultrafiltration involves:

(a) adding an acid to a solution which contains a protein associated with a cofactor which is to be released under acidic conditions and applying them to ultrafiltration to collect a non-permeate; and

(b) adding a further acid to the collected non-permeate and applying them to ultrafiltration to collect a further non-permeate, wherein the step (b) is carried out at lease once.

In another embodiment, the protein is lactoferrin, and the acid is citric acid.

In yet another embodiment, the concentration of the acid is 0.01 to 1 mol/L.

The invention also provides an apparatus for producing an apoprotein, which includes a tank for receiving a starting material solution, means for feeding an acid, an ultrafiltration module provided with an ultrafiltration membrane, and means for removing a permeate.

In one embodiment, the apparatus further includes a tank for collecting a non-permeate.

### BRIEF DESCRIPTION OF DRAWINGS

FIG. 1 is a diagram that illustrates the method of the invention.

### BEST MODE FOR CARRYING OUT THE INVENTION

### Protein Associated with Cofactor

According to the present invention, any proteins may be used as a starting material for the production of apoprotein as far as the protein is associated with a cofactor which is to be released under acidic conditions (holoprotein, including enzymes). Examples of the protein include hemes, amylases, hexokinases, and metal proteases, more specifically including lactoferrin, transferrin, ferritin, iron-associated protein derived from egg, hemoglobin, myoglobin, and cytochrome. Examples of the cofactor include prosthetic groups, including flavin adenine dinucleotide (FAD), heme, and flavin mononucleotide (FMN); coenzymes, including thiamine diphosphate, pyridoxal phosphate, nicotinamide adenine dinucleotide (NAD), nicotinamide adenine dinucleotide phosphate (NADP), and coenzyme A (CoA); and metal ions, including ferrous, copper, manganese, zinc, cobalt, vanadium, and calcium ions.

If the protein, which is associated with a cofactor which is to be released under acidic conditions, is lactoferrin, then the cofactor is generally metal ions (in particular, ferrous ion).

According to the method of the invention, a solution which contains a protein associated with a cofactor which is to be released under acidic conditions can be used as a starting material. The solution of starting material can be any solutions without restrictions as far as the solution contains a protein associated with a cofactor which is to be released under acidic conditions. It is preferable that the solution does not include any substances having a molecular weight greater than that of the protein. Inorganic salts and substances having a molecular weight lower than that of the protein in the solution can be removed by ultrafiltration.

There are no particular restrictions on how the solution of starting material is obtained or prepared. The solution may be a solution having dissolved an isolated and/or purified product of protein which is naturally occurring or which has been produced by genetic engineering. The solution may be also a solution having dissolved a commercially-available protein. For example, in the case of lactoferrin, the solution of lactoferrin can be obtained from whey by, for example, the adsorption onto a cation exchange resin and subsequent desorption with a high-concentration salt solution, the separation by electrophoresis, or the separation by affinity chromatography.

### (Acid)

There are no particular restrictions on the acid for use in the present invention, as long as the acid can cause the release of cofactor and pass through the membrane along with the cofactor to be removed away under ultrafiltration. Preferably, examples of the acid include inorganic acids such as hydrochloric acid, sulfuric acid, nitric acid, phosphoric acid, and carbonic acid; and organic acids such as acetic acid, benzoic acid, and citric acid. The acid for use can be appropriately selected depending on proteins of interest. For example, in the case of lactoferrin, preferably the acid is citric acid, hydrochloric acid, or phosphoric acid, and particularly preferably citric acid.

There also are no particular restrictions on the concentration of acid to be added to the solution, but it is likely that the aqueous solution of acid at a higher concentration will denature the protein whereas the aqueous solution of acid at a lower concentration cannot result in the desired acidic conditions efficiently. Thus, preferably, the concentration of acid is from 0.001 mol/L, more preferably 0.01 mol/L, further more preferably 0.03 mol/L, even further more preferably 0.05 mol/L, to 10 mol/L, more preferably 5 mol/L, further more preferably 1 mol/L, even further more preferably 0.5 mol/L. The amount of acid to be added may be dependent on proteins of interest. It is sufficient to add the acid to the range of pH where the protein can release its cofactors. For example, in the case of lactoferrin, it is preferable that the acid (in particular, acids as listed above) is added at an appropriate concentration to adjust a pH within 0.5 to 3.5, and the amount of acid to be added is preferably 1 mol/L or less, more preferably 0.01 to 1 mol/L.

### (Production of Apoprotein)

According to the present invention, it is important to add an acid in the process of ultrafiltration (hereinafter, which may be also referred to as “process of acid-added ultrafiltration”). If the addition of acid and the ultrafiltration are carried out completely independently, the desired effect will not be probably obtained. For example, when a solution, in which cofactor(s) have been already released from a protein by adding an acid (i.e., which already has an apoprotein and the released cofactor(s)), is applied to ultrafiltration, the apoprotein will reassociate with the released cofactor prior to the passage of the solution through an ultrafiltration membrane, and the reassociated protein will remain in a non-permeate. Therefore, a holoprotein, which is associated with cofactor(s), will remain in a non-permeate, thus preventing the efficient production of apoprotein.

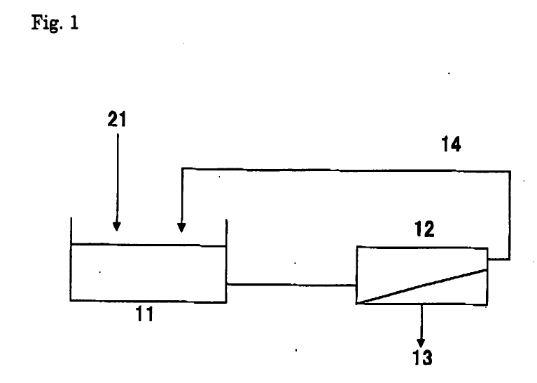

There are no particular restrictions on the process of acid-added ultrafiltration, as long as the process is such that the acid is added in the process of ultrafiltration so that the protein of interest can be concentrated. The process of acid-added ultrafiltration can be a batch-type or continuous-type, and either may be used. The process may be appropriately chosen depending on factors such as the purpose of separation, the amount to be processed, and the characteristics of a solution of starting material (hereinafter, which is referred to as “starting material solution”). The batch processing is preferable. The procedure is explained in greater detail with reference to FIG. 1.

For example, in the case of the batch processing, an acid 21 is added to a starting material solution that has been received into a tank 11 and they are applied to an ultrafiltration membrane (ultrafiltration module 12) to remove a permeate 13 out of the system and collect a non-permeate 14 in a tank (not shown in FIG. 1). The non-permeate 14 collected in a reduced volume by ultrafiltration is received into the tank 11, and a further acid 21 (preferably the same acid as above) is added thereto and they are applied to ultrafiltration. The procedure as immediately above can be repeated until the concentration of apoprotein and the rate of released cofactor will arrive at those to be intended. In view of the efficiency for collecting an apoprotein, the number of times that the procedure (addition of acid plus ultrafiltration) is performed is appropriately two to five times, but not be limited thereto. For example, the addition of acid 21 to the starting material solution and the passage of the starting material solution through the ultrafiltration membrane (ultrafiltration module 12) can be carried out contiguously or simultaneously so that any cofactors released by the addition of acid will not reassociate with the apoprotein. On the other hand, the apoprotein is concentrated in the non-permeate.

In the case of the continuous processing, a starting material solution is applied to the ultrafiltration membrane (ultrafiltration module 12), and a non-permeate 14 which contains the protein is received back to the tank 11 and recirculated, during which an acid 21 is added quantitatively to the circulation line, such as the tank 11. In view of the concentration, the amount of acid to be added should be less than the amount of permeation.

According to the method, due to the addition of acid 21, the cofactor is released, and the released cofactor passes through the ultrafiltration membrane along with the acid and thus is removed out of the system, whereas the apoprotein, which has released its cofactor, does not pass through the ultrafiltration membrane and thus is efficiently concentrated in the non-permeate.

The temperature of ultrafiltration is within the range of usually 5 to 70° C., and preferably 10 to 40° C. It is not preferable when the temperature is higher or lower than them. When the temperature is higher, the protein may be easily denatured. On the other hand, when the temperature is lower, the amount of permeation through membrane may be decreased to reduce the efficiency for concentration.

Although the thus obtained concentrate would contain the apoprotein and the acid, the acid can be removed by performing further ultrafiltration with a suitable solvent such as water. In order to wash the ultrafiltration membrane, further ultrafiltration may be performed with a suitable solvent such as water.

According to the present invention, an apparatus can be used for producing an apoprotein, which includes a tank 11 for receiving a starting material solution, means (not shown) for feeding an acid 21, an ultrafiltration module 12 provided with an ultrafiltration membrane, and means (not shown) for removing a permeate 13, and optionally, a tank (not shown) for collecting a non-permeate 14. These parts can be connected by predetermined pipelines. The acid 21 can be added into a tank 11 for receiving a starting material solution, by any means capable of feeding the acid in a batch or continuous manner. The liquid (such as a starting material solution or acid 21) received into the tank 11 can be applied to the ultrafiltration module 12 by pumping. A tank for receiving a permeate 13 may be also included in order to remove the permeate 13 out of the system of the addition of acid plus ultrafiltration. The apparatus can be additionally provided with valves, measuring instruments such as flow meters or pressure meters, frames, or distribution panels. Accessory elements required for membrane washing, etc. may be added to the apparatus as necessary. There are no particular restrictions on the apparatus for use in ultrafiltration, and any commercially available apparatuses may be used.

There are no particular restrictions on the ultrafiltration membrane for use in the ultrafiltration module 12, and the membrane may be appropriately selected depending on the molecular weight and the structure of the protein serving as a starting material. The molecular weight cut off of 3,000 to 100,000, which can be a molecular weight cut off of the ultrafiltration membrane commonly used, can be useful. It is not suitable when the molecular weight cut off is smaller or larger than them. When the molecular weight cut off is smaller, it may be time consuming for filtration. When the molecular weight cut off is larger, it may result in the lose of proteins. For example, in order to obtain a concentrate of apolactoferrin from lactoferrin with a molecular weight of approximately 80,000, the molecular weight cut off can be selected to be 5,000 to 80,000.

Examples of the material for the ultrafiltration membrane include organic membranes made of natural or synthetic polymers such as cellulose acetate, polysulfone, polyethersulfone, polyacrylamide, polyimide, aromatic polyamide, polyacrylonitrile, and hydrophilic polyolefines; and inorganic membranes made of ceramics such as alumina, zirconia, and titanium.

The type of the membrane includes hollow fiber module, flat sheet module, and flat membrane. In view of filtration rates, the type of hollow fiber module is preferably selected.

By removing solvent from the thus obtained apoprotein solution, optionally using a concentrator such as an evaporator, a freeze-dry lyophilizer, or a spray drier, the apoprotein can be obtained.

According to the method of the invention, the apoprotein can be produced at high purity without contaminations, for example, of microorganisms. Accordingly, the apoprotein produced according to the method of the invention can be used as a raw material in various fields, including food, drugs, and cosmetics.

### EXAMPLES

The following examples are provided in order to describe the invention in more specific detail, but the invention should not be limited to the examples.

### Example 1

### Batch Processing

Into the bench filtration apparatus for pencil modules (from Asahi Kasei Chemicals; PS-24001), the ACP-0013 UF module (from Asahi Kasei Chemicals, hollow-fiber module, membrane inner diameter of 0.8 mm, effective membrane area of 170 cm², membrane made of polyacrylonitrile, nominal molecular weight cut off of 18,000) was incorporated to prepare the ultrafiltration apparatus for use in the example.

First, 1000 mL of a 100 mg/mL hololactoferrin (100% ferrous bound: ferric chloride was added to lactoferrin from Sigma, and the unbound iron was removed by dialysis) aqueous solution was received at room temperature into the feed tank of the ultrafiltration apparatus, whose operation start pressure was set to a module output pressure of 50 KPa, and applied to ultrafiltration for allowing the solution to be concentrated and reducing its volume to 500 mL. Next, to this solution, a 0.05 mol/L hydrochloric acid aqueous solution was added until a solution volume reached 1000 mL, and immediately after, they were applied to ultrafiltration for allowing the solution to be concentrated and reducing its volume to 500 mL. The procedure as above was repeated once again, and then a 0.05 mol/L hydrochloric acid aqueous solution was added to this solution until a solution volume reached 1000 mL, and immediately after, they were applied to ultrafiltration for allowing the solution to be concentrated and reducing its volume to 250 mL. The finally obtained concentrated solution was taken, and the amount of iron bound to lactoferrin was measured at an absorbance of 470 nm to determine the rate of released iron from lactoferrin. The concentration factor of the resulting solution was approximately four times, and the rate of released iron from lactoferrin was 74%.

### Example 2

### Continuous Processing

Using the same apparatus as in Example 1, 1000 mL of a 100 mg/mL hololactoferrin (100% iron bound) aqueous solution was received at room temperature into the feed tank whose operation start pressure has been set to 50 KPa for the module output pressure, and while continuously feeding a 0.05 mol/L hydrochloric acid aqueous solution at 6 ml/min, ultrafiltration was conducted for three hours, yielding a solution at a volume of 280 mL. The concentrated solution was taken, and the amount of iron bound to lactoferrin was measured at an absorbance of 470 nm to determine the rate of released iron from lactoferrin. The concentration factor of the resulting solution was approximately 3.6 times, and the rate of released iron from lactoferrin was 68%.

### Example 3

### Evaluation of Apolactoferrin for Denaturation

Into the bench filtration apparatus for pencil modules (Microza®UF·MF; PS-24001; Asahi Kasei Chemicals), the AHP-0013 UF module (from Asahi Kasei Chemicals, hollow fiber module, membrane inner diameter of 0.8 mm, effective membrane area of 170 cm², membrane made of polyacrylonitrile, nominal molecular weight cut off of 50,000) was incorporated to prepare the ultrafiltration apparatus for use.

First, 1000 mL of a 2 wt % hololactoferrin (30% iron bound: purity degree of 90%: Fonterra Co-operative Group) aqueous solution was received at room temperature into the feed tank of the ultrafiltration apparatus, whose operation start pressure was set to 50 KPa for the module output pressure, and applied to ultrafiltration. The ultrafiltration was carried out in the same manner as in Example 1, except that a solution of 0.1 mol/L or 1 mol/L of citric acid, hydrochloric acid, or nitric acid was added instead of the 0.05 mol/L hydrochloric acid aqueous solution. More specifically, first, the lactoferrin solution was applied to ultrafiltration for allowing the solution to be concentrated and reducing its volume to 500 mL. Next, to the resultant solution, one of the above solutions of acid was added until a solution volume reached 1000 mL, and immediately after, they were applied to ultrafiltration for allowing the solution to be concentrated and reducing its volume to 500 mL. The same procedure was repeated once again, and then the same solution of acid was added until a solution volume reached 1000 mL, and immediately after, they were applied to ultrafiltration for allowing the solution to be concentrated and reducing its volume to 250 mL. Through ultrafiltrations, the finally obtained solution was concentrated from the starting material solution by a factor of three.

The thus obtained concentrate was freeze-dried to obtain a powdered product. The powdered product was dissolved in pure water, and then applied to the antibody assay using BIOXYTECH® Lacto f EIA™ (OXIS International Inc., Oregon, USA). The rate for response to the lactoferrin antibody was determined to evaluate whether or not lactoferrin was denatured by the processing as explained above. The results are shown in Table 1.

As understood from Table 1 below, in the cases of using 1 mol/L acid solutions, the rate for response to antibody was low and in part, denaturation of lactoferrin was observed. In particular, in the case of using 1 mol/L of nitric acid, protein aggregations were observed, and the characteristic of lactoferrin was not exhibited.

TABLE 1

| Concentration | | Hydrochloric | |
|---|---|---|---|
| of acid | Citric acid | acid | Nitric acid |
| 1 mol/L | 61% | 57% | 0% |
| 0.1 mol/L | 100% | 100% | 93% |

### Example 4

### Evaluation for the Rate of Apoprotein Production

The process of ultrafiltration of a lactoferrin aqueous solution was performed as in Example 3 except that a 0.1 mol/L citric acid solution was selected as the acid. As a control, the process was also performed using a pure water at least 18 M Ω·cm (prepared using Milli-Q (Academic A10; Millipore Corporation)) in lieu of the acid. Through ultrafiltrations, the finally obtained solution was concentrated from the starting material solution by a factor of three.

The production of apoprotein from lactoferrin was evaluated based on the content of iron. The resultant concentrate was freeze-dried to obtain a powdered product. Then, the powdered product was dissolved in a 0.1 mol/L hydrochloric acid aqueous solution to be a 3 wt % (apo)lactoferrin solution. Then, the concentration of iron in this solution was measured with the atomic absorption spectrophotometer (AAnlyst 400; Perkin Elmer), and the production of apoprotein was evaluated.

The results are shown in Table 2 below.

TABLE 2

| | Treatment | | |
|---|---|---|---|
| | | Number of | |
| | Number of | additions of | |
| | additions of acid | water | Fe(ppm) |
| Lactofarrin solution | 0 | 0 | 2.348 |
| Apolactoferrin solution | 1 | 0 | 2.049 |
| obtained following | 1 | 1 | 2.017 |
| addition of 0.1 mol/L | 1 | 2 | 1.966 |
| citric acid or water | 2 | 0 | 1.427 |
| | 3 | 0 | 1.104 |
| | 4 | 0 | 0.832 |
| | 5 | 0 | 0.597 |
| | 0 | 1 | 2.444 |
| | 0 | 2 | 2.607 |
| | 0 | 3 | 2.610 |
| | 0 | 4 | 2.727 |
| | 0 | 5 | 2.582 |

As clear from Table 2, the production of apoprotein from lactoferrin was developed with the increase in the number of additions of citric acid. On the other hand, any effect on the production of apoprotein was not observed with the addition of water.

### Example 5

### Evaluation for the Industrially Scale Manufacturing of Apolactoferrin

Into the microza UF for laboratory use (LX-22001; Asahi Kasei Chemicals), the LOV UF module (from Asahi Kasei Chemicals, hollow fiber module, membrane inner diameter of 0.8 mm, effective membrane area of 41 m², membrane made of polyacrylonitrile, nominal molecular weight cut off of 50,000) was incorporated to prepare the ultrafiltration apparatus for use.

Using 8.98 kg of 20 mg/mL lactoferrin solution, apolactoferrin was produced as follows. The order of additions of a 0.1 mol/L citric acid solution and a pure water at least 18 M Ω·cm in the manufacturing of apolactoferrin is as shown in Table 9. In the ultrafiltration using this apparatus, the lactoferrin solution was received into the feed tank of the apparatus, and was circulated for ten minutes, and was then flowed in the reverse direction for five seconds, for allowing the solution to be concentrated. The same procedure was repeated until the unpermeated concentrate was reduced by half (this was regarded as one round). Then, the citric acid solution was feeded to the tank instead of the lactoferrin solution, and the same procedure as above was carried out for two rounds. Then, water was feeded to the tank, and the above procedure was repeated for three rounds to remove away the remaining acid in the unpermeated concentrate. The process of manufacturing yielded 4.04 kg of the concentrate which contains apolactoferrin.

This concentrate was freeze-dried to obtain a powdered product which was white. The purity of apolactoferrin in the product was 85.3%. The identification and the measurement for purity of apolactoferrin were carried out by the antibody assay using BIOXYTECH® Lacto f EIA™ (OXIS International Inc., Oregon, USA). The powdered product was dissolved in water, yielding a 2 wt % solution with a pH of 3.05.

The 2 wt % solution was diluted with water by a factor of 100, and was evaluated for the contamination of microorganisms by determining antimicrobial activities of the solution by the microplating. The procedure was performed as below. The 100-times diluted solution (1 mL) was introduced into a well, and then the SCD broth (1 mL) was introduced into the well at the twice concentration and incubated for three days at 35° C. After the incubation, the growth of microorganisms was visually examined in the well. The following results were obtained: 0 CFU/g for the contamination of bacteria, and negative for E. coli, staphylococci, salmonella strains, molds, and yeasts.

In order to evaluate the operability of the ultrafiltration apparatus in the manufacturing of apolactoferrin, the input and output pressures of the UF membrane, the circulating flow rate, and the permeate flow rate were also measured. These measurements were performed at the passage of the lactoferrin solution alone (the first group of rows in Table 3; this is indicated in Table 3 by 20 mg/mL LF solution at UF 50,000 concentration), the addition of citric acid for a first time (the second group of rows in Table 3; this is indicated in Table 3 by 38.4 mg/mL CA-added UF permeation (1)), the addition of citric acid for a second time (the third group of rows in Table 3; this is indicated in Table 3 by 38.4 mg/mL CA-added UF permeation (2)), the addition of water for a first time (the fourth group of rows in Table 3; this is indicated in Table 3 by water-added UF permeation (1)), the addition of water for a second time (the fifth group of rows in Table 3; this is indicated in Table 3 by water-added UF permeation (2)), and the addition of water for a third time (the sixth group of rows in Table 3; this is indicated in Table 3 by water-added UF permeation (3)). The results are shown in Table 8.

Next, the UF membrane used for the manufacturing of apolactoferrin was washed as shown in Table 4 with an aqueous solution containing sodium hydroxide (indicated in Table 4 by NaOH) at 3% (weight/volume) and 5% sodium hypochlorite (indicated in Table 4 by NaOCl) at 0.6% (volume/volume), and the input and output pressures of the UF membrane, the circulating flow rate, and the permeate flow rate were measured. The results are shown in Table 4.

TABLE 3

| | | | | | Circulation | | UF input |
|---|---|---|---|---|---|---|---|
| | Concentrate | Permeate | Time | | time | flow time | pressure PI-1 |
| Step | (kg) | (kg) | Time | Minutes | (minutes) | (seconds) | (Mpa) |
| 20 mg/mL LF solution | 8.98 | — | 9:22 | 0 | 10 | 5 | 0.128 |
| at UF50,000 | — | 1.49 | 9:37 | 15 | 10 | 5 | 0.128 |
| concentration | — | 4.00 | 9:52 | 30 | 10 | 5 | 0.128 |
| | — | 4.62 | 9:59 | 37 | 10 | 5 | 0.127 |
| 38.4 mg/mL CA added | +4.50 | — | 10:04 | 0 | 10 | 5 | 0.126 |
| UF permeation (1) | — | 1.61 | 10:19 | 15 | 10 | 5 | 0.127 |
| | — | 3.57 | 10:34 | 30 | 10 | 5 | 0.127 |
| | — | 4.71 | 10:41 | 37 | 10 | 5 | 0.128 |
| 38.4 mg/mL CA added | +4.48 | — | 10:44 | 0 | 10 | 5 | 0.127 |
| UF permeation (2) | — | 1.54 | 10:59 | 15 | 10 | 5 | 0.127 |
| | — | 3.36 | 11:14 | 30 | 10 | 5 | 0.127 |
| | — | 4.51 | 11:20 | 36 | 10 | 5 | 0.128 |
| water added | +4.50 | — | 11:23 | 0 | 10 | 5 | 0.126 |
| UF permeation (1) | — | 1.41 | 11:38 | 15 | 10 | 5 | 0.126 |
| | — | 3.92 | 11:53 | 30 | 10 | 5 | 0.126 |
| | — | 4.52 | 11:59 | 36 | 10 | 5 | 0.127 |
| water added | +4.50 | — | 12:01 | 0 | 10 | 5 | 0.124 |
| UF permeation (2) | — | 1.66 | 12:16 | 15 | 10 | 5 | 0.125 |
| | — | 4.53 | 12:37 | 36 | 10 | 5 | 0.125 |
| water added | +4.50 | — | 12:39 | 0 | 10 | 5 | 0.124 |
| UF permeation (2) | — | 1.86 | 12:54 | 15 | 10 | 5 | 0.124 |
| | — | 4.51 | 13:15 | 36 | 10 | 5 | 0.125 |

| | |
|---|---|
| ALF concentrate | 4.04 |

| | | | | | | Circulating |
|---|---|---|---|---|---|---|
| | Con- | UF output | Circulating | Permeate | Circulating | pump |
| | centrate | pressure PI- | flow rate FI- | flow rate FI- | temperature | frequency |
| Step | kg | 2 (Mpa) | 1 (L/min) | 2 (L/min) | (° C.) | (Hz) |
| 20 mg/mL LF solution | 8.98 | 0.078 | 15.00 | 0.138 | 11.4 | 40 |
| at UF50,000 | — | 0.078 | 15.00 | 0.110 | 15.3 | 40 |
| concentration | — | 0.078 | 15.00 | 0.096 | 19.5 | 40 |
| | — | 0.077 | 14.25 | 0.069 | 21.7 | 40 |
| 38.4 mg/mL CA added | +4.50 | 0.081 | 15.00 | 0.138 | 16.5 | 40 |
| UF permeation (1) | — | 0.081 | 15.00 | 0.110 | 19.6 | 40 |
| | — | 0.079 | 15.00 | 0.082 | 23.3 | 40 |
| | — | 0.078 | 14.25 | 0.096 | 25.2 | 40 |
| 38.4 mg/mL CA added | +4.48 | 0.083 | 15.00 | 0.138 | 19.0 | 40 |
| UF permeation (2) | — | 0.081 | 15.00 | 0.138 | 22.0 | 40 |
| | — | 0.080 | 15.00 | 0.110 | 25.1 | 40 |
| | — | 0.078 | 14.25 | 0.096 | 26.3 | 40 |
| water added | +4.50 | 0.083 | 15.00 | 0.151 | 18.9 | 40 |
| UF permeation (1) | — | 0.081 | 15.00 | 0.138 | 22.0 | 40 |
| | — | 0.080 | 15.00 | 0.110 | 25.5 | 40 |
| | — | 0.080 | 15.00 | 0.096 | 26.5 | 40 |
| water added | +4.50 | 0.082 | 15.00 | 0.151 | 19.2 | 40 |
| UF permeation (2) | — | 0.081 | 15.00 | 0.124 | 22.3 | 40 |
| | — | 0.080 | 15.00 | 0.096 | 26.7 | 40 |
| water added | +4.50 | 0.083 | 15.00 | 0.151 | 20.1 | 40 |
| UF permeation (2) | — | 0.083 | 15.00 | 0.138 | 23.0 | 40 |
| | — | 0.083 | 15.00 | 0.082 | 27.2 | 40 |
| ALF concentrate | 4.04 | | | | | |

TABLE 4

| | | | | | | flow | | | | Permeate | | Circulating |
|---|---|---|---|---|---|---|---|---|---|---|---|---|
| | | 5% | | | Circulation | time | UF input | UF output | Circulating | flow rate | Circulating | pump |
| | | NaClO | Time | | time | (sec- | pressure PI-1 | pressure PI- | flow rate FI- | FI- | temperature | frequency |
| Step | NaOH % | ppm | Time | Minutes | (minutes) | onds) | (Mpa) | 2(Mpa) | 1 (L/min) | 2 (L/min) | (° C.) | (Hz) |
| UF | 0.3 | 300 | 13:57 | 0 | 10 | 5 | 0.122 | 0.078 | 24.00 | 0.110 | 13.1 | 40 |
| wash | (15 | (30 | 14:27 | 30 | 10 | 5 | 0.125 | 0.078 | 28.50 | 0.234 | 22.8 | 40 |
| | g/5 L) | mL/5 L) | 14:57 | 60 | 10 | 5 | 0.124 | 0.079 | 28.50 | 0.331 | 27.1 | 40 |
| | | | 15:27 | 90 | 10 | 5 | 0.123 | 0.077 | 27.75 | 0.717 | 32.2 | 40 |
| | | | 15:57 | 120 | 10 | 5 | 0.122 | 0.076 | 27.75 | 1.186 | 35.0 | 40 |
| | | | 16:27 | 150 | 10 | 5 | 0.122 | 0.076 | 27.75 | 1.366 | 35.4 | 40 |
| | | | 16:57 | 180 | 10 | 5 | 0.123 | 0.076 | 27.75 | 1.449 | 37.1 | 40 |

During the manufacturing of apolactoferrin, no change was observed in the input and output pressures of the UF membrane and the circulating flow rate. On the other hand, it could be found that as the concentration proceeded, the permeate flow rate was decreased to reduce the efficiency for processing, like in the ultrafiltration membrane processing of other biological substances.

In the membrane wash following the manufacturing of apolactoferrin, no change was observed in the input and output pressures of the UF membrane and the circulating flow rate. On the other hand, it could be found that as the washing proceeded, the permeate flow rate was increased to develop the membrane wash.

Thus, in this example, apolactoferrin could be produced without the loss of the operability of the ultrafiltration apparatus.

### INDUSTRIAL APPLICABILITY

According to the method of the invention, by adding an acid to a solution which contains a protein and applying the solution to ultrafiltration, the cofactors released from the protein are continuously removed away by passing through the ultrafiltration membrane along with the acid, whereas the resultant apoprotein remains in the concentrate without passing through the ultrafiltration membrane. Therefore, the resultant apoprotein is prevented from reassociating with the cofactor, which makes the production of apoprotein efficiently. The resultant apoprotein can be concentrated simultaneously, which allows the process for production of apoprotein to be simplified.

1. A method for producing an apoprotein, comprising:
adding an acid to a solution which contains a protein associated with a cofactor which is to be released under acidic conditions and applying them to ultrafiltration.

2. The method according to claim 1, wherein the method comprises:
(a) adding an acid to a solution which contains a protein associated with a cofactor which is to be released under acidic conditions and applying them to ultrafiltration to collect a non-permeate; and
(b) adding a further acid to the collected non-permeate and applying them to ultrafiltration to collect a non-permeate, wherein the step (b) is carried out at least once.

3. The production method according to claim 1, wherein the protein is lactoferrin, and the acid is citric acid.

4. The production method according to claim 3, wherein a concentration of the acid is 0.01 to 1 mol/L.

5. An apparatus for producing an apoprotein, comprising:
a tank for receiving a starting material solution;
means for feeding an acid;
an ultrafiltration module provided with an ultrafiltration membrane; and
means for removing a permeate.

6. The apparatus according to claim 5, further comprising:
a tank for collecting a non-permeate.

7. The production method according to claim 2, wherein the protein is lactoferrin, and the acid is citric acid.

8. The production method according to claim 7, wherein a concentration of the acid is 0.01 to 1 mol/L.
